(19) United States
(12) Patent Application Publication
(10) Pub. No.: US 2023/0172878 A1
(43) Pub. Date: Jun. 8, 2023

(54) COMPOSITIONS AND METHODS FOR TREATING CYTOKINE STORMS

(71) Applicant: Annette M. Tobia, Wyndmoor, PA (US)

(72) Inventor: Annette M. Tobia, Wyndmoor, PA (US)

(21) Appl. No.: 17/921,071

(22) PCT Filed: Apr. 30, 2021
(86) PCT No.: PCT/US2021/030224

§ 371 Date: Oct. 24, 2022

Related U.S. Application Data
(60) Provisional application No. 63/018,308, filed on Apr. 30, 2020.

(51) Int. Cl. A61K 31/133; A61P 37/02

(52) U.S. Cl. CPC A61K 31/133; A61P 37/02

(57) ABSTRACT

A composition for, and method of, treating a condition in a subject wherein said condition is brought about, at least in part, by a cytokine storm in the subject, comprising administering to the subject said composition which comprises meglumine or a salt thereof.

### BACKGROUND OF THE INVENTION

Cytokines play an important role in normal immune responses but having a large amount of them released in the body all at once can be harmful. Such a severe immune reaction in which the body releases too many cytokines into the blood too quickly is referred to as a “Cytokine Storm.” (URL is cancer.gov/publications/dictionaries/cancer-terms/def/797584) A cytokine storm can occur as a result of a wide variety of infectious and noninfectious diseases, an autoimmune condition, or other disease. It may also occur after treatment with some types of immunotherapy. In particular, cytokine storms can also be the unintended result of therapeutic actions such as chimeric antigen receptor (“CAR”) T cell therapy. (URL is blog.dana-farber.org/insight/2017/08/what-are-the-side-effects-of-car-t-cell-therapy/, accessed 4/9/2020).

The cytokine storm has captured the attention of the public and the scientific community alike, and the general notion of an excessive or uncontrolled release of proinflammatory cytokines is well known. (Tisoncik, Jennifer R et al. “Into the eye of the cytokine storm.” Microbiology and molecular biology reviews: MMBR vol. 76, 1 (2012): 16-32. doi:10.1128/MMBR.05015-11). Cytokines are inflammatory immunologic proteins that are there to fight off infections and ward off cancers, that, when out of control, can make one very ill.” (Cron, Cytokine Storm Syndrome, Springer, 2019).

The NCI Dictionary of Cancer Terms describes a cytokine storm as a severe immune reaction in which the body releases too many cytokines into the blood too quickly. In cytokine storms large numbers of white blood cells are activated and release inflammatory cytokines, which in turn activate yet more white blood cells. The signs and symptoms of a cytokine storm include high fever, inflammation (redness and swelling), and severe fatigue and nausea. Sometimes, a cytokine storm may be severe or life threatening and lead to multiple organ failure.

Other names for cytokine storm are hypercytokinemia; cytokine release syndrome (“CRS”), macrophage activation syndrome (“MAS”), and hemophagocytic lymphohistiocytosis. When occurring as a result of drug administration, cytokine storms are also known as infusion reactions. (URL is newswise.com/articles/here-s-a-playbook-for-stopping-deadly-cytokine-storm-syndrome accessed 0/05/2020)

The term “cytokine storm” conjures images of an immune system gone awry and an inflammatory response that is out of control. However, there is not a good understanding of the molecular events that precipitate a cytokine storm, of the contribution such a “storm” makes to pathogenesis, or of what therapeutic strategies might be used to prevent the storm or quell it once it has started. (Tisoncik et al., op cit.).

The first use of “cytokine storm” appears to be in an article published in 1993 on graft-versus-host disease. The use of the term in infectious disease research began in early 2000 in reports on cytomegalovirus, Epstein-Barr virus-associated hemophagocytic lymphohistiocytosis, group A streptococcus, influenza virus, variola virus, and severe acute respiratory syndrome coronavirus (“SARS-CoV”). The term appears to have first been applied in the context of avian H5N1 influenza virus infection in 2005.

Cytokine storms are initiated when large numbers of white blood cells are activated and release inflammatory cytokines. These cytokines, in turn, activate yet more white blood cells. Cytokine storms are also an adverse effect of some monoclonal antibody drugs, as well as adoptive T-cell therapies.

The development of Macrophage Activation Syndrome (“MAS”), an episode of overwhelming inflammation that occurs most commonly in children with systemic juvenile idiopathic arthritis, is characterized by a cytokine storm. (Schulert, Grant S, and Alexei A Grom. “Macrophage activation syndrome and cytokine-directed therapies.” Best Practice & Research. Clinical Rheumatology vol. 28,2 (2014): 277-92. doi:10.1016/j.berh.2014.03.002).

### Cytokines

Cytokines are a diverse group of proteins secreted by cells for intercellular signaling and communication. Specific cytokines have autocrine, paracrine, and/or endocrine activity and, via binding to receptors, elicit a variety of responses. Their responses depend upon the cytokine and the target cell. Among cytokines' functions are control of cell proliferation and differentiation and the regulation of angiogenesis and immune and inflammatory responses. Many cytokines have multiple and sometimes unrelated functions. Table 1 lists the various cytokines and their respective actions.

TABLE 1
Major Types and Actions of Cytokines

| Type | Actions |
|---|---|
| Interferons | Regulation of innate immunity, activation of |
| | antiviral properties, antiproliferative effects |
| Interleukins | Growth and differentiation of leukocytes; many are |
| | proinflammatory |
| Chemokines | Control of chemotaxis, leukocyte recruitment; many |
| | are proinflammatory |
| Colony-stimulating | Stimulation of hematopoietic progenitor cell |
| factors | proliferation and differentiation |
| Tumor necrosis | Proinflammatory, activates cytotoxic T lymphocytes |
| factor | |

### Interferons

Interferons (“IFN”s) play a central role in innate immunity to viruses and other microbial pathogens. They are classified into three major types (types I, II, and III) on the basis of their receptor specificity. Interferons' receptor binding initiates downstream signaling cascades, which activate transcription factors and the induction of hundreds of IFN-stimulated genes to encode protein products with antiviral, antiproliferative, or immunomodulatory properties.

### Interleukins

The interleukins are a diverse family of immune system regulators that function primarily in immune cell differentiation and activation. Interleukins may be either pro- or anti-inflammatory and elicit a wide variety of responses.

Proinflammatory cytokines, IL-1α and IL-1β, mediate the host response to infection through direct and indirect mechanisms. These cytokines increase acute-phase signaling, trafficking of immune cells to the site of primary infection, epithelial cell activation, and secondary cytokine production. Inflammasomes are cytosolic macromolecular complexes comprised of members of the nucleotide-binding domain and leucine-rich-repeat-containing receptor (NLR) family (the NLRP3 inflammasome is one the best characterized) that produce IL-1β and IL-18 in defense against pathogens. The acute-phase response to infection results in a wide range of local effects and systemic alterations that are evidenced in changes that are generally proinflammatory, such as the increase in specific cytokine production, and which can be linked to viral clearance. IL-1 receptor signaling is responsible for acute lung immunopathology but increases the survival of mice infected with influenza virus by enhancing IgM antibody responses and recruiting CD4+ T cells to the site of infection.

### Chemokines

The largest family of cytokines is the chemokines. Chemokines bind to one or more G-protein-coupled receptors. Chemokines function as chemoattractants to control the migration of cells, particularly those of the immune system. Chemokines also aid in other processes, such as embryogenesis, innate and adaptive immune system development and function, and cancer metastasis. Most chemokines are considered proinflammatory and are released by a variety of cells in response to virus (or other microbial) infection. The release of proinflammatory chemokines results in immune system cell (neutrophils, monocytes/macrophages, and lymphocytes) recruitment to the site of infection. Chemokines and their receptors have been heavily targeted by the pharmaceutical industry, but with limited success.

### CSFs

Colony-stimulating factors (“CSF”s) stimulate hematopoietic progenitor cell proliferation and differentiation. CSFs are of three types: granulocyte-macrophage colony-stimulating factor (GM-CSF), macrophage colony-stimulating factor (M-CSF), and granulocyte colony-stimulating factor (G-CSF). CSFs are also associated with inflammation. There is evidence that CSFs may be part of a mutually dependent proinflammatory cytokine network that includes IL-1 and tumor necrosis factor (“TNF”).

### TNFs

TNF is now considered a central cytokine in acute viral diseases, including those caused by influenza virus, dengue virus, and Ebola virus. TNF is expressed by a variety of immune cells, and its primary receptor, TNFR1, appears to be expressed by all cell types, ensuring widespread effects of this cytokine. Excess TNF production is associated with many chronic inflammatory and autoimmune diseases, and TNF inhibitors have been approved for the treatment of inflammatory bowel disease, psoriasis, and rheumatoid arthritis. In contrast, the use of TNF inhibitors for the treatment of sepsis has not yet proven successful.

### Cytokine Storm Pathology

Cytokine storm-initiated inflammation starts at a local site and spreads via systemic circulation, resulting in redness, swelling or edema, heat, pain, and loss of function at the local site. These responses increase blood flow, enable vascular leukocytes and plasma proteins to reach extravascular sites of injury, causing the above-listed factors, thereby warning of the local responses. Repair processes are then initiated to restores tissue and organ function. With severe inflammation, repair of local tissue structures results in healing with fibrosis, which may result in persistent organ dysfunction.

Acute lung injury (“ALI”) is a common consequence of a cytokine storm in the lung alveolar environment and systemic circulation. ALI is associated with suspected or proven infections in the lungs or other organs. In humans, ALI is characterized by an acute mononuclear/neutrophilic inflammatory response followed by a chronic fibroproliferative phase marked by progressive collagen deposition in the lung. Pathogen-induced lung injury can progress into ALI or its more severe form, acute respiratory distress syndrome (“ARDS”), as seen with SARS-CoV, Coronavirus Disease 2019 (“COVID-19”), and influenza virus infections.

The cytokine storm is exemplified by severe lung infections, in which local inflammation spills over into the systemic circulation, producing systemic sepsis, as defined by persistent hypotension, hyper- or hypothermia, leukocytosis or leukopenia, and often thrombocytopenia. Viral, bacterial, and fungal pulmonary infections all cause the sepsis syndrome, and these etiological agents are difficult to differentiate on clinical grounds. In some cases, persistent tissue damage without severe microbial infection in the lungs also is associated with a cytokine storm and clinical manifestations that mimic sepsis syndrome. In addition to lung infections, the cytokine storm is a consequence of severe infections in the gastrointestinal tract, urinary tract, central nervous system, skin, joint spaces, and other sites.

Symptoms include high fever, enlarged spleen, excessive bleeding, low counts of all types of blood cells (red, white, and platelets) and, potentially, multiple organ failures. (Newswise.com, op.cit.).

### Causes

Cytokine storms are associated with a wide variety of infectious and noninfectious diseases. Randy Cron, M.D., Ph.D., professor of pediatrics and medicine at the University of Alabama-Birmingham stated that “there are 80 to 100 bugs that have been associated with it in case reports.” (Newswise.com, op. cit.). Cytokine storms have also been the unfortunate consequence of attempts at therapeutic intervention.

The following bacterial infections are among those associated with cytokine storms: streptococcus, staphylococcus, and pancreatitis.

Viral infections associated with cytokine storms include influenza viruses (including, without limitation, H1N1, H5N1, wine influenza, Spanish influenza, and avian (bird) influenzas), human corona viruses (including, without limitation, coronaviruses 229E, NL63, OC43, HKU1, MERS-CoV, SARS-CoV, and SARS-CoV2), other non-human corona viruses, other viruses (including, without limitation, Epstein Barr virus (including the strain causing mononucleosis), virus variola, dengue virus, and Ebola virus, West Nile virus, hepatitis A virus, hepatitis B virus, hepatitis C virus, respiratory viruses, herpes, cytomegalovirus (“CMV”) and any mutation of any of the foregoing.

Non-infectious conditions can bring about cytokine storms (including, without limitation, graft-versus-host disease, (“GVHD”), multiple sclerosis, multiple organ dysfunction syndrome, rheumatic diseases, such as juvenile arthritis and lupus, fibrosis, lung injuries and blood cancers, like leukemias and lymphomas).

Therapeutic activity may result in cytokine storms. Surface receptors on T cells can cause a cytokine storm when activated by therapeutic monoclonal antibodies (“mAb”s). Recently, cytokine storms also have been noted in 20% to 30% of cases of patients treated with CAR-T as immunotherapy for cancer. Certain patients on heart-lung bypass machines, such as extracorporeal membrane oxygenation devices, are beset with cytokine storms. (Newswise.com. op. cit.).

Certain genetic mutations are risk factors for mortality from cytokine storms. (Schulert, Grant S et al. “Whole-Exome Sequencing Reveals Mutations in Genes Linked to Hemophagocytic Lymphohistiocytosis and Macrophage Activation Syndrome in Fatal Cases of H1N1 Influenza.” The Journal of infectious diseases vol. 213,7 (2016): 1180-8. doi:10.1093/infdis/jiv550).

### Current Treatments

Liao, et al. disclose the use of meglumine as an additive to cyclic adenosine monophosphate (“cAMP”), the additive being in the form of meglumine cyclic adenylate (MCA). However, MCA used as a permeability enhancer, not for its therapeutic effect. (Liao, J., Xie, J., Lin, D., Lu, N., Guo, L., Li, W., Pu, B., Yang, Y., Yang, Z., Zhang, Y., Song, Y. “Meglumine cyclic adenylate improves neurological function following acute spinal cord injury in rats.” Molecular Medicine Reports 10.3 (2014): 1225-1230. Moreover, Liao et al. report negative coronary side effects of MCA. (Jia L, Hou D H. Zhongguo Yao Li Xue Bao. 1988; 9(5):421-426).

U.S. Patent Application Publication No. 2014/0275237 to Faulds et al. discloses using a Beraprost isomer in the treatment of a pathology associated with a cytokine storm. In certain embodiments the Beraprost isomer is in the form of a salt made with a cationic portion. Faulds discloses using meglumine as one of many possible choices for the cation in such salts, however there is no teaching of any particular therapeutic benefit of having meglumine for therapeutic purpose, nor even is there any suggestion of having meglumine as a preferred cation among the 13 disclosed cations.

Researchers report the use of dasatinib (Sprycel), a type of tyrosine kinase inhibitor approved for the treatment of leukemia, in temporarily shutting down cytokine storms resulting from CAR-T without destroying the cells. The drug works by blocking the phosphorylation step of CAR-T cell activation. However, their research required prompt administration of the drug. Moreover, the drug comes at great monetary cost to the subject and carries dangerous side effects, limiting its use. (URL is mskcc.org/blog/drug-hits-car-snooze-button-can-quiet-cytokine-storm 1) (URL is www.webmd.com/drugs/2/drug-144641/sprycel-oral/details 2).

Patients suffering cytokine storms in relation to COVID-19 recovered after receiving IV infusions of the rheumatoid arthritis drug Actemra, a brand of tocilizumab. Acterma blocks the cytokine IL-6 receptor, one of several that soar in the COVID-19 cytokine storm. In some cases these Acterma treatments are supplemented with a large vitamin C injection (4,000 to 5,000 times the normal amount in the bloodstream). (URL is richmond.com/special-report/coronavirus/a-richmond-doctor-s-dramatic-story-of-covid-19-infection-hospitalization-and-survival/article 750722ad-7918-544d-bc4d-798d456033f6.html, accessed 4-20-2020; URL is webmd.com/lung/news/20200417/cytokine-storms-may-be-fueling-some-covid-deaths, accessed 4-19-2020).

It may be possible to head off the cytokine storm altogether by blocking some of the chemicals that can trigger its release, one type of which are catecholamines. Catecholamines enhance inflammatory injury by augmenting the production of IL-6 and other cytokines through a self-amplifying feed-forward loop in immune cells. In a non-reviewed study, the medical records of more than 12,673 people with acute respiratory distress syndrome, or ARDS, the same diagnosis given to many of severely ill COVID-19 patients. These patients were not infected with the virus that causes COVID-19, however. The data revealed that patients taking medications that block the release of catecholamines in the year before their diagnosis were about 20% less likely to need to be placed on a ventilator after their diagnosis, compared to others, an effect that was statistically significant. Prophylactic inhibition of catecholamine synthesis was achieved with the al-AR antagonist prazosin. Prazosin reduces catecholamines and cytokine responses in mice and resulted in markedly increased survival following various hyper-inflammatory stimuli. (Konig, et al., “Targeting the catecholamine-cytokine axis to prevent SARS-CoV-2 cytokine storm syndrome” (URL doi.org/10.1101/2020.04.02.20051565).

The safety and efficacy of use of the drugs as a method of alleviating cytokine storms is experimental, and the cases don't really provide solid scientific information about whether the drugs work the way we think they should, or offer any guidance about when they should be used. Moreover, the drugs may prove too costly for widespread use. For example, Actemra is expensive, costing between $3,000 and $5,000 per dose, and its supply may be limited.

There is a need in the art for novel compositions for, and methods of, treating a condition in a subject wherein said condition is brought about, at least in part, by a cytokine storm in the subject. The present invention satisfies this need.

### SUMMARY OF THE INVENTION

One aspect of the invention features a method of treating a disorder in a subject wherein said disorder is brought about, at least in part, by a cytokine storm in the subject, wherein the method comprises administering to the subject a composition that comprises meglumine or a salt thereof.

The method of the invention further comprises administering to the subject a composition which comprises meglumine or a salt thereof, and a therapeutic agent selected from the group consisting of pain medications, antibiotics, antiviral medications, and anti-inflammatory agents.

The method of the invention further includes administering to the subject the composition which comprises meglumine or a salt thereof and further comprises administering to the subject a therapeutic agent is selected from the group consisting of those medications used to alleviate the disorder causing the cytokine storm. In one embodiment, the disorder causing the cytokine storm is selected from the group consisting of bacterial infections, viral infections, non-infectious conditions, genetic mutations, and therapeutic activity. In one embodiment, the bacterial infection is selected from the group consisting of streptococcus, staphylococcus, pancreatitis, and any mutation of any of the foregoing. In one embodiment, the viral infection is selected from the group consisting of influenza viruses, human corona viruses, other non-human corona viruses, Eptein Barr virus (including the strain causing mononucleosis), virus variola, dengue virus, Ebola virus, West Nile virus, hepatitis A virus, hepatitis B virus, hepatitis C virus, respiratory viruses, herpes cytomegalovirus, and any mutation of any of the foregoing. In one embodiment, the influenza virus is selected from the group consisting of H1N1, H5N1, wine influenza, Spanish influenza, avian (bird) influenzas, and any mutation of any of the foregoing. In one embodiment, the corona virus is selected from the group consisting of 229E, NL63, OC43, HKU1, MERS-CoV, SARS-CoV, SARS-CoV2, and any mutation of any of the foregoing. In one embodiment, the non-infectious condition is selected from the group consisting of graft-versus-host disease, (“GVHD”), multiple sclerosis, multiple organ dysfunction syndrome, fibrosis, lung injuries, rheumatic diseases, and blood cancers. In one embodiment, the rheumatic disease is selected from the group consisting of juvenile arthritis and lupus. In one embodiment, the blood cancer is selected from the group consisting of leukemia and lymphoma. In one embodiment, the therapeutic activity is selected from the group consisting of mAb therapy and CAR-T.

One aspect of the invention features a composition for treating a condition in a subject wherein said condition is brought about, at least in part, by a cytokine storm in the subject, wherein said composition comprises meglumine or a salt thereof.

The composition of the invention comprises meglumine or a salt thereof. In one embodiment, the meglumine or salt thereof is the only active ingredient in the composition. In another embodiment, the composition further comprises a therapeutic agent selected from the group consisting of pain medications, antibiotics, antiviral medications, and anti-inflammatory agents.

The composition of the invention comprises a composition for treating a condition in a subject wherein said condition is brought about, at least in part, by a cytokine storm in the subject wherein said cytokine storm is caused by an external event, wherein said external event is selected from the group consisting of bacterial infections, viral infections, non-infectious conditions, genetic mutations, and therapeutic activity. In one embodiment, the bacterial infection is selected from the group consisting of streptococcus, staphylococcus, pancreatitis, and any mutation of any of the foregoing. In one embodiment, the viral infection is selected from the group consisting of influenza viruses, human corona viruses, other non-human corona viruses, Epstein Barr virus (including the strain causing mononucleosis), virus variola, dengue virus, Ebola virus, West Nile virus, hepatitis A virus, hepatitis B virus, hepatitis C virus, respiratory viruses, herpes, cytomegalovirus, and any mutation of any of the foregoing. In one embodiment, the influenza virus is selected from the group consisting of H1N1, H5N1, wine influenza, Spanish influenza, avian (bird) influenzas, and any mutation of any of the foregoing. In one embodiment, the corona virus is selected from the group consisting of 229E, NL63, OC43, HKU1, MERS-CoV, SARS-CoV, SARS-CoV2, and any mutation of any of the foregoing. In one embodiment, the non-infectious condition is selected from the group consisting of graft-versus-host disease, (“GVHD”), multiple sclerosis, multiple organ dysfunction syndrome, fibrosis, lung injuries, rheumatic diseases, and blood cancers. In one embodiment, the rheumatic disease is selected from the group consisting of juvenile arthritis and lupus. In one embodiment, the blood cancer is selected from the group consisting of leukemia and lymphoma. In one embodiment, the therapeutic activity is selected from the group consisting of mAb therapy and CAR-T.

The meglumine or a salt thereof of the invention further comprises a composition comprising a therapeutic agent selected from the group consisting of time release agents, permeation enhancers, pharmaceutically acceptable carriers, and controlled-release components.

The method of the invention further includes administering to the subject the composition which comprises meglumine or a salt thereof by a route selected from the group consisting of inhalational, oral, rectal, vaginal, parenteral, topical, transdermal, pulmonary, intranasal, buccal, ophthalmic, intrathecal, parenteral, intravenous, subcutaneous, intramuscular, and any combinations thereof.

The method of the invention wherein the composition comprising meglumine or a salt thereof is administered to the subject in a therapeutically effect amount. The invention further comprises a method wherein the therapeutically effective amount is greater than 4 grams of meglumine or a salt thereof per subject per day. In one embodiment the method of the invention comprises a method wherein the therapeutically effective amount is between about 5 grams of meglumine or a salt thereof grams per subject per day and about 100 grams of meglumine or a salt thereof per subject per day. In one embodiment the method of the invention comprises a method wherein the therapeutically effective amount is between about 10 grams of meglumine or a salt thereof grams per subject per day and about 80 grams of meglumine or a salt thereof per subject per day. In one embodiment the method of the invention comprises a method wherein the therapeutically effective amount is about 15 grams per subject per day. The invention further comprises the therapeutically effective amount being about 500 mg of meglumine or a salt thereof per kg of body weight of the subject and about 80 mg of meglumine or a salt thereof per kg of body weight of the subject. The invention further comprises the therapeutically effective amount being about 70 mg of meglumine or a salt thereof per kg of body weight of the subject. A therapeutically effective amount includes a dose wherein 70 kg subject receives 15 grams of meglumine or a salt thereof per day.

In one embodiment, the invention comprises administering meglumine, or a salt thereof, in an amount equal to the amount of meglumine contained in an accepted dosage of DOTAREM® (gadoterate-meglumine). DOTAREM is currently FDA-approved for intravenous administration at a dose that contains between about 0.9 grams and about 10 grams of meglumine (ionically bound to the gadoterate) in a 10 ml solution, i.e., between about 90 mg and about 100 mg per ml of meglumine. The invention does not require the administration of the gadoterate portion of DOTAREM.

In one embodiment, the invention comprises the intravenous administration of meglumine or a salt thereof at between an amount between about 0.2 grams to about 10 grams per dose. Such a dose could be administered during a period of once per hour to about once per six hour period.

In one embodiment, the invention comprises the oral administration of meglumine or a salt thereof at a release level of between an amount between about 0.5 grams to about 3 grams per patch.

In one embodiment, the invention comprises the transdermal administration of meglumine or a salt thereof at between an amount between about 0.2 grams to about 4 grams per dose.

The invention further comprises composition for, and method of, treating a condition in a subject wherein said condition is brought about, at least in part, by a cytokine storm in the subject, wherein the method comprises administering to the subject said composition which comprises meglumine or a salt thereof, wherein said cytokine storm arises in connection with another disease or disorder. In one embodiment the other disease or disorder is selected from the group consisting of bacterial infections, viral infections, non-infectious conditions, genetic mutations, and therapeutic activity. In one embodiment, the bacterial infection is selected from the group consisting of streptococcus, staphylococcus, pancreatitis, and any mutation of any of the foregoing. In one embodiment, the viral infection is selected from the group consisting of influenza viruses, human corona viruses, other non-human corona viruses, Epstein Barr virus (including the strain causing mononucleosis), virus variola, dengue virus, Ebola virus, West Nile virus, hepatitis A virus, hepatitis B virus, hepatitis C virus, respiratory viruses, herpes, cytomegalovirus, and any mutation of any of the foregoing. In one embodiment, the influenza virus is selected from the group consisting of H1N1, H5N1, wine influenza, Spanish influenza, avian (bird) influenzas, and any mutation of any of the foregoing. In one embodiment, the corona virus is selected from the group consisting of 229E, NL63, OC43, HKU1, MERS-CoV, SARS-CoV, SARS-CoV2, and any mutation of any of the foregoing. In one embodiment, the non-infectious condition is selected from the group consisting of graft-versus-host disease, (“GVHD”), multiple sclerosis, multiple organ dysfunction syndrome, fibrosis, lung injuries, rheumatic diseases, and blood cancers. In one embodiment, the rheumatic disease is selected from the group consisting of juvenile arthritis and lupus. In one embodiment, the blood cancer is selected from the group consisting of leukemia and lymphoma. In one embodiment, the therapeutic activity is selected from the group consisting of mAb therapy and CAR-T.

### BRIEF DESCRIPTION OF THE DRAWINGS

FIG. 1 shows the structure of meglumine.

For FIGS. 2 through 7, cells from two donors were used, each in a separate independent study. Cytokines secreted in the media by the cells were measured at 2, 4, 6, 24 and 48 hours.

FIGS. 2A and 2B show IL-6 production being decreased in response to the addition of various concentrations of meglumine.

FIGS. 3A and 3B show TNF-α production being decreased in response to the addition of various concentrations of meglumine.

FIGS. 4A and 4B show MIP 1α production being decreased in response to the addition of various concentrations of meglumine.

FIGS. 5A and 5B show MCP 1 production being decreased in response to the addition of various concentrations of meglumine.

FIGS. 6A and 6B show IL-10 production being decreased in response to the addition of various concentrations of meglumine.

FIG. 7 shows IL-6 production being stimulated by incubation of human cells with 40 mM morpholinofructose.

FIG. 8 shows the reduction of IL-6 in cells where IL-6 production was stimulated by the addition of lipopolysaccharide (“LP S”).

FIG. 9 shows the increase of IL-1α by the addition of meglumine to cells where IL-1α production was stimulated by the addition of LPS.

FIG. 10 shows the reduction of IL-1β by the addition of meglumine to cells where IL-1B production was stimulated by the addition of LPS.

FIG. 11 shows the reduction of IL-8 by the addition of higher doses of meglumine to cells where IL-8 production was stimulated by the addition of LPS.

FIG. 12 shows the reduction of IL-9 by the addition of higher doses of meglumine to cells where IL-9 production was stimulated by the addition of LPS.

FIG. 13 shows the reduction of MIP-1α by the addition of higher doses of meglumine to cells where MIP-1α production was stimulated by the addition of LPS.

FIG. 14 shows the reduction of MIP-1β by the addition of higher doses of meglumine to cells where MIP-1β production was stimulated by the addition of LPS.

FIG. 15 shows the reduction of vascular endothelial growth factor (“VEGF”) by the addition of higher doses of meglumine to cells where VEGF production was stimulated by the addition of LPS.

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

As used herein, “alleviating a disease or disorder symptom,” means reducing the severity of the symptom.

A “compound” or “composition” mean any type of substance or agent that is commonly considered a drug, or a candidate for use as a drug, as well as combinations and mixtures of the above, or modified versions or derivatives of the compound.

The term “controlled-release component” means a compound or compounds, including, but not limited to, polymers, polymer matrices, gels, permeable membranes, liposomes, or microspheres or a combination thereof that facilitates the controlled-release of the active ingredient.

A “cytokine storm” is a severe immune reaction in which the body releases too many cytokines into the blood too quickly. Cytokine storms are also known as “hypercytokinemia,” “cytokine release syndrome” (“CRS”), “macrophage activation syndrome” (“MAS”), and “hemophagocytic lymphohistiocytosis.” When occurring as a result of drug administration, cytokine storms are also known as “infusion reactions.”

A “disease” is a state of health of an animal wherein the animal cannot maintain homeostasis, and wherein if the disease is not ameliorated then the animal's health continues to deteriorate. As used herein, normal aging is included as a disease.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“DOTAREM” means DOTAREM® (gadoterate-meglumine) as described at URL accessdatafda.gov/drugsatfda_docs/label/2018/204781s0081bl.pdf.

An “effective amount” or “therapeutically effective amount” of a compound is that amount of compound which is sufficient to provide a beneficial effect to the subject to which the compound is administered or gives the appearance of providing a therapeutic effect as in a cosmetic.

“Intramuscular” or “IM” means within or into muscle. An “intramuscular injection” is an injection into a muscle.

“Intravenous” or “IV” means a way of giving a drug or other substance through a needle or tube inserted into a vein.

A “mutation” is any change in the DNA sequence of a cell. A “mutant” is an organism that has undergone a mutation. A “virus mutant” or “virus mutation” or “viral mutant” or a “mutation of a virus” is a virus that is made by the mutation of a progeny virus.

A “peripherally inserted central catheter” or “PICC” is a device used to draw blood and give treatments, including intravenous fluids, drugs, or blood transfusions. A thin, flexible tube is inserted into a vein in the upper arm and guided (threaded) into the superior vena cava. A needle is inserted into a port outside the body to draw blood or give fluids therethrough. A peripherally inserted central catheter may stay in place for extended time periods, avoiding the need for repeated needle sticks.

“Permeation enhancement” and “permeation enhancers” as used herein relate to the process and added materials that bring about an increase in the permeability of skin or other biological barrier (including a cell membrane or a barrier to the blood stream) to a poorly permeating pharmacologically active agent, i.e., so as to increase the rate at which the drug permeates through the barrier and enters the area on the opposite side of the barrier. “Permeation enhancer” is used interchangeably with “penetration enhancer”.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

A “prophylactic” treatment is a treatment administered to a subject who does not exhibit signs of a disease or exhibits only early signs of the disease for the purpose of decreasing the risk of developing pathology associated with the disease.

“Subcutaneous” means beneath the skin. A “subcutaneous port” is a tube surgically placed into a blood vessel and attached to a disk placed under the skin to administer intravenous fluids and drugs or obtain blood samples.

A “therapeutic” treatment is a treatment administered to a subject who exhibits signs of pathology, for the purpose of diminishing or eliminating those signs.

By “transdermal” delivery is intended both transdermal (or “percutaneous”) and transmucosal administration, i.e., delivery by passage of a drug through the skin or mucosal tissue and into the bloodstream. Transdermal also refers to the skin as a portal for the administration of drugs or compounds by topical application of the drug or compound thereto.

The term “topical application”, as used herein, refers to administration to a surface, such as the skin. This term is used interchangeably with “cutaneous application”.

The term to “treat,” as used herein, means reducing the frequency with which symptoms are experienced by a patient or subject or administering an agent or compound to reduce the frequency with which symptoms are experienced.

As used herein, “treating a disease or disorder” means reducing the frequency with which a symptom of the disease or disorder is experienced by a patient. Disease and disorder are used interchangeably herein. As used herein, the term “wild-type” refers to the genotype and phenotype that is characteristic of most of the members of a species occurring naturally and contrasting with the genotype and phenotype of a mutant.

### About Meglumine

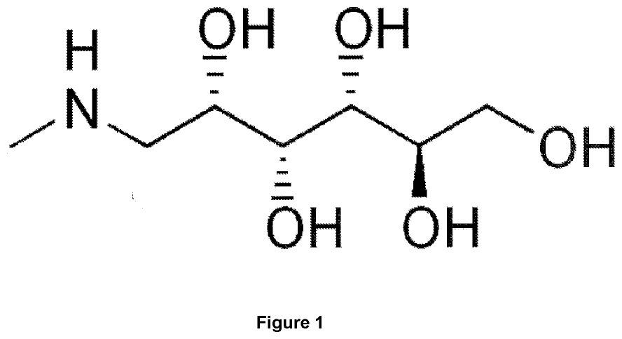

Meglumine is a sugar alcohol derived from glucose. It is also derived from sorbitol. Meglumine's IUPAC name is (2R,3R,4R,5S)-6-(Methylamino)hexane-1,2,3,4,5-pentol. Other names for meglumine include N-methyl-D-glucamine; Methylglucamine; N-Methylglucamine; 1-Deoxy-1-(methylamino)-D-glucitol; 1-Deoxy-1-methylaminosorbitol; N- and Methylsorbitylamine. The structure of meglumine is shown in FIG. 1.

Sugar alcohols, like xylitol and sorbitol, are food ingredients that are “Generally Regarded As Safe” (“GRAS”) by the FDA. Meglumine is currently used for non-therapeutic purposes as an excipient. No adverse side effects of meglumine have ever been reported.

Meglumine, an FDA-approved pharmaceutical excipient, has been in use for over 50 years and is safe even at the highest doses. Searching the FDA database, Inactive Ingredient Search for Approved Drug Products, (URL: www.accessdata.fda.gov/scripts/cder/iig/index.Cfm) gives the following listing in response to a search for meglumine:

TABLE 2
Pharmaceutical Uses of Meglumine

| | | | | | Maximum | |
|---|---|---|---|---|---|---|
| Inactive | | | CAS | | Potency per | |
| Ingredient | Route | Dosage Form | Number | UNII | unit dose | |
| Meglumine | Intramuscular | Injection | 6284408 | 6HG8UB2MUY | 14% | |
| Meglumine | Intravascular | Injection | 6284408 | 6HG8UB2MUY | ADJ PH | |
| Meglumine | Intravenous | Injection | 6284408 | 6HG8UB2MUY | 15.4% | |
| Meglumine | Iv(Infusion) | Injection | 6284408 | 6HG8UB2MUY | 5.7% | |
| Meglumine | Oral | Capsule, Delayed | 6284408 | 6HG8UB2MUY | 3.5 | MG |
| | | Action | | | | |
| Meglumine | Oral | Capsule, Delayed | 6284408 | 6HG8UB2MUY | NA | |
| | | Action, Enteric | | | | |
| | | Coated | | | | |
| Meglumine | Oral | Capsule, Delayed | 6284408 | 6HG8UB2MUY | 3.5 | MG |
| | | Release | | | | |
| Meglumine | Oral | Capsule, Enteric | 6284408 | 6HG8UB2MUY | 3 | MG |
| | | Coated Pellets | | | | |
| Meglumine | Oral | Capsule, Extended | 6284408 | 6HG8UB2MUY | 5 | MG |
| | | Release | | | | |
| Meglumine | Oral | Tablet | 6284408 | 6HG8UB2MUY | 24 | MG |
| Meglumine | Oral | Tablet (Immed./ | 6284408 | 6HG8UB2MUY | 1 | MG |
| | | Comp. Release), | | | | |
| | | Film Coated | | | | |
| Meglumine | Oral | Tablet, Delayed | 6284408 | 6HG8UB2MUY | 0.5 | MG |
| | | Action | | | | |
| Meglumine | Oral | Tablet, Delayed | 6284408 | 6HG8UB2MUY | 10 | MG |
| | | Release | | | | |

| | | | | | |
|---|---|---|---|---|---|
| Meglumine | Oral | Tablet, Film | 6284408 | 6HG8UB2MUY | NA |
| | | Coated | | | |
| Meglumine | Ureteral | Solution | 6284408 | 6HG8UB2MUY | 7% |

FDA-approved imaging agents, such as DOTAREM, contain an acute bolus dose of 16 grams of meglumine (230 mg/kg). Meglumine-containing contrast agents are sold under many trade names, including DOTAREM and Clariscan. These agents are Gadolinium-based contrast agent for use in magnetic resonance imaging of the brain, spine, and associated tissues to detect and visualize areas with disruption of the blood brain barrier and/or abnormal vascularity. They are normally dispensed as IV solutions at concentration of 0.5 mmol/mL (which contains 376.9 mg/mL gadoterate meglumine). (Fallenberg, E. M., Renz, D. M., Karle, B. et al. “Intraindividual, randomized comparison of the macrocyclic contrast agents gadobutrol and gadoterate meglumine in breast magnetic resonance imaging.” Eur Radiol 25, 837-849 (2015) (URL is doi.org/10.1007/s00330-014-3426-0). DOTAREM is currently FDA-approved for intravenous administration at a dose that contains approximately 0.9 grams of meglumine (ionically bound to the gadoterate) in a 10 ml solution. The invention does not require the administration of the gadoterate portion of DOTAREM.

Data summarized in a separate Meglumine Safety report show meglumine HCl is safe in rats, mice, and dogs at 10×higher doses than the human equivalent dose (HED) levels proposed for clinical trials.

The invention relates to the administration of an identified compound in a pharmaceutical composition to practice the methods of the invention, the composition comprising the compound or an appropriate derivative or fragment of the compound and a pharmaceutically acceptable carrier.

In one embodiment, the pharmaceutical compositions useful for practicing the invention may be administered to deliver a dose of between 1 ng/kg of subject body mass/day and 100 g/kg/day. In another embodiment, the pharmaceutical compositions useful for practicing the invention may be administered to deliver a dose of between 1 ng/kg/day and 100 g/kg/day. The method of the invention wherein the composition comprising meglumine or a salt thereof is administered to the subject in a therapeutically effect amount. The invention further comprises a method wherein the therapeutically effective amount is greater than 4 grams of meglumine or a salt thereof per subject per day. In one embodiment the method of the invention comprises a method wherein the therapeutically effective amount is between about 5 grams of meglumine or a salt thereof grams per subject per day and about 100 grams of meglumine or a salt thereof per subject per day. In one embodiment the method of the invention comprises a method wherein the therapeutically effective amount is between about 10 grams of meglumine or a salt thereof grams per subject per day and about 80 grams of meglumine or a salt thereof per subject per day. In one embodiment the method of the invention comprises a method wherein the therapeutically effective amount is about 15 grams per subject per day. The invention further comprises the therapeutically effective amount being about 500 mg of meglumine or a salt thereof per kg of body weight of the subject and about 80 mg of meglumine or a salt thereof per kg of body weight of the subject. The invention further comprises the therapeutically effective amount being about 70 mg of meglumine or a salt thereof per kg of body weight of the subject. A therapeutically effective amount includes a dose wherein 70 kg subject receives 15 grams of meglumine or a salt thereof per day. The pharmaceutical compositions useful for practicing the invention may be administered to deliver a dose of about 0.9 grams of meglumine in a 10 ml solution.

In one embodiment, the meglumine is formulated in a liquid pharmaceutical composition. This can be used for intravenous injection or infusion. The formulation can contain a range of concentrations of meglumine, e.g., at least about 10 mg, or at least about 20 mg, or at least about 30 mg, or at least about 40 mg, or at least about 50 mg, or at least about 60 mg, or at least about 70 mg, or at least about 80 mg, or at least about 90 mg, or about at least 100 mg, or at least about 110 mg, or at least about 120 mg, or at least about 130 mg, or at least about 140 mg, or at least about 150 mg, or at least about 160 mg, or at least about 170 mg, or at least about 180 mg, or at least about 190 mg, or at least about 200 mg, or more per mL, up to the solubility limit for a given solvent or buffer. Upper limits may be up to about 10 mg, or up to about 20 mg, or up to about 30 mg, or up to about 40 mg, or up to about 50 mg, or up to about 60 mg, or up to about 70 mg, or up to about 80 mg, or up to about 90 mg, or up to about 100 mg, or up to about 110 mg, or up to about 120 mg, or up to about 130 mg, or up to about 140 mg, or up to about 150 mg, or up to about 160 mg, or up to about 170 mg, or up to about 180 mg, or up to about 190 mg, or up to about 200 mg, or more per mL, up to the solubility limit for a given solvent or buffer. In one embodiment, the formulation contains between about 10 mg and about 200 mg per mL, or between about 20 mg and about 190 mg per mL, or between about 30 mg and about 180 mg per mL, or between about 40 mg and about 170 mg per mL, or between about 50 mg and about 160 mg per mL, or between about 60 mg and about 150 mg per mL, or between about 70 mg and about 140 mg per mL, or between about 80 mg and about 130 mg per mL, or between about 90 mg and about 120 mg per mL. In one embodiment, the formulation contains between about 90 mg and about 100 mg of meglumine per mL.

The dosage range for administration of a liquid formulation of meglumine via intravenous injection or infusion can vary. In one embodiment, a bolus dose of, e.g., about 10 mg, or about 20 mg, or about 30 mg, or about 40 mg, or about 50 mg, or about 60 mg, or about 70 mg, or about 80 mg, or about 90 mg, or about 100 mg, or about 120 mg, or about 130 mg, or about 140 mg, or about 150 mg, or about 160 mg, or about 170 mg, or about 180 mg, or about 190 mg, or about 200 mg per kilogram of body weight is administered.

In one embodiment, the pharmaceutical composition comprises the meglumine in a physiologically acceptable carrier, such as buffered saline or other buffered aqueous solution. In one embodiment, the pharmaceutical composition contains no active ingredients other than the meglumine.

As will be understood by the skilled artisan, when armed with the disclosure set forth herein, a composition useful in the present invention can include one active ingredient, i.e., meglumine, as stated above. Alternatively, a composition useful in the present invention can include at least two active ingredients. In one aspect, multiple active ingredients may be active in an additive manner. In another aspect, multiple active ingredients may be active in a synergistic manner. That is, the multiple active ingredients in a composition of the invention may provide a therapeutic effect that is greater than the addition of the therapeutic effects provided by each of the active ingredients alone. By way of a non-limiting example, a composition can comprise both the meglumine or salt thereof and an additional compound for treating the cytokine storm or the accompanying disorders, such as congestion, dizziness, nausea, and the like.

Other pharmaceutically acceptable carriers that are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water, saline, or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like. In one embodiment the composition is administered intravenously. In one intravenous administration, the composition could be administered as a contrast agent in therapeutically effective amounts. The recommended dose of DOTAREM is, for adult and pediatric patients (including term neonates), 0.2 mL/kg (0.1 mmol/kg) body weight administered as an intravenous bolus injection, manually or by power injector, at a flow rate of approximately 2 mL/second for adults and 1-2 mL/second for pediatric patients. DOTAREM is currently FDA-approved for intravenous administration at a dose that contains 0.9 grams of meglumine (ionically bound to the gadoterate) in a 10 ml solution. The invention does not require the administration of the gadoterate portion of DOTAREM.

To ensure complete injection of DOTAREM the injection may be followed by normal saline flush. The method of the invention further comprises administering composition of the invention with another pharmaceutical compositions or additional ingredients. In an intravenous administration the composition of the invention could be delivered via IV injection, either one currently in place, or in one specifically used to administer the composition of the invention.

Pharmaceutical compositions that are useful in the methods of the invention may be administered systemically in oral solid formulations, ophthalmic, suppository, aerosol, topical or other similar formulations. In addition to the compound of the invention, other compounds, such as heparan sulfate, or a biological equivalent thereof, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer compounds according to the methods of the invention.

Modification of pharmaceutical compositions suitable for administration to humans in order to render the compositions suitable for administration to various animals is well understood, and the ordinarily skilled pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gel caps, and caplets, that are adapted for controlled release are encompassed by the present invention.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

As used herein, an “oily” liquid is one which comprises a carbon-containing liquid molecule and which exhibits a less polar character than water.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, a paste, a gel, a toothpaste, a mouthwash, a coating, an oral rinse, or an emulsion. The terms oral rinse and mouthwash are used interchangeably herein.

A pharmaceutical composition of the invention may be prepared, packaged, or sold in a formulation suitable for oral or buccal administration. Such a formulation may comprise, but is not limited to, a gel, a liquid, a suspension, a paste, a toothpaste, a mouthwash or oral rinse, and a coating. For example, an oral rinse of the invention may comprise a compound of the invention at about 1.4%, chlorhexidine gluconate (0.12%), ethanol (11.2%), sodium saccharin (0.15%), FD&C Blue No. 1 (0.001%), peppermint oil (0.5%), glycerine (10.0%), Tween 60 (0.3%), and water to 100%. In another embodiment, a toothpaste of the invention may comprise a compound of the invention at about 5.5%, sorbitol, 70% in water (25.0%), sodium saccharin (0.15%), sodium lauryl sulfate (1.75%), carbopol 934, 6% dispersion in (15%), oil of spearmint (1.0%), sodium hydroxide, 50% in water (0.76%), dibasic calcium phosphate dihydrate (45%), and water to 100%. The examples of formulations described herein are not exhaustive and it is understood that the invention includes additional modifications of these and other formulations not described herein, but which are known to those of skill in the art.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated, or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water, saline, or 1,3-butane diol, for example. Other acceptable diluents and solvents include, but are not limited to, Ringer's solution, isotonic sodium chloride solution, and fixed oils such as synthetic mono- or di-glycerides. Other parentally administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### Examples

In the following examples cells were stimulated known cytokine stimulants, either lipopolysaccharide (“LPS”) or morpholino fructose. The response for IL-6 production for morpholinofructose is shown in FIG. 7 (human PBMCs incubated with 40 mM morpholinofructose produce significantly more IL-6 than control cells). Varying levels of meglumine, or a salt thereof were then administered to the stimulated cells and the resulting cytokine levels measured.

### Example 1

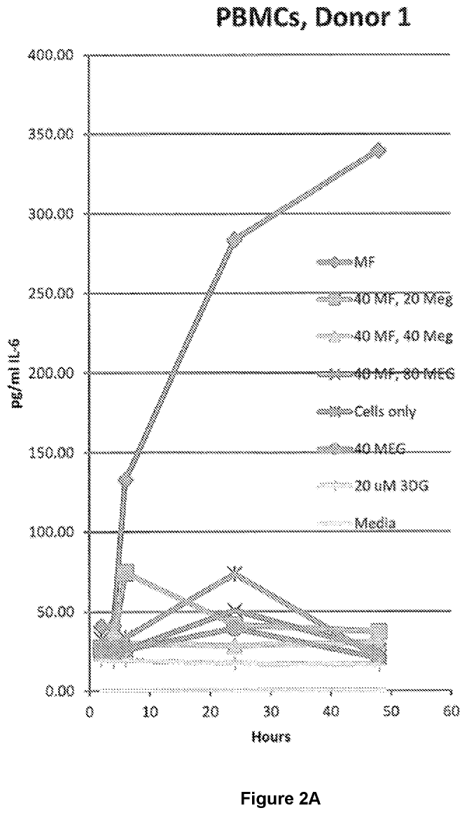

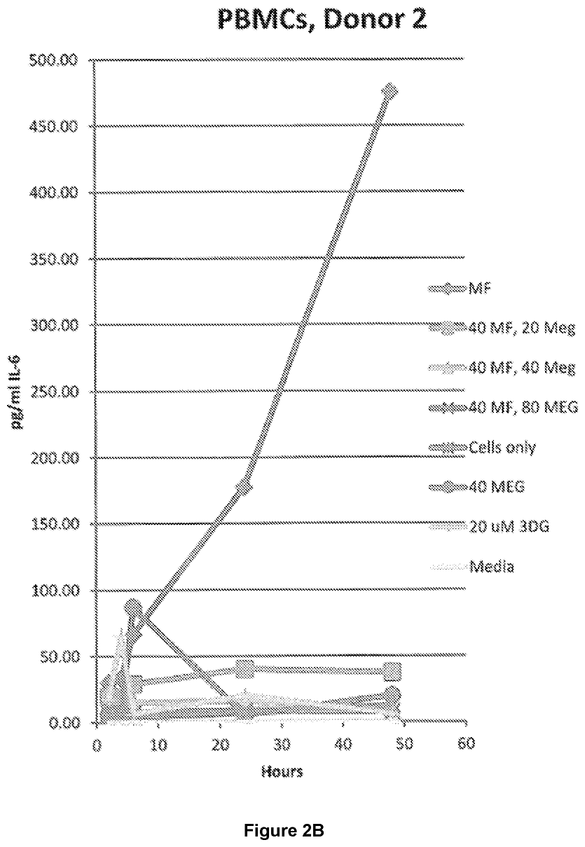

Peripheral blood mononuclear cells (“PBMC”s) from two donors were used to measure cytokine levels in response to various inputs. For each donor, one group of PBMCs were untreated. One group was allowed to remain in cell culture. A different group was treated with 40 mM meglumine. Another group was treated with morpholinofructose at 40 mM concentration. Three remaining groups were treated with morpholinofructose at 40 mM concentration and meglumine at concentration of 20 mM, 40 mM, and 80 mM. The groups were allowed to incubate for up to 50 hours. Cytokines secreted in the media by the cells were measured at 2, 4, 6, 24 and 48 hours The percentage of cells positive for IL-6 was determined by flow cytometry. The results are shown in FIGS. 2A and 2B. The addition of meglumine reduced the IL-6 levels in the PBMCs.

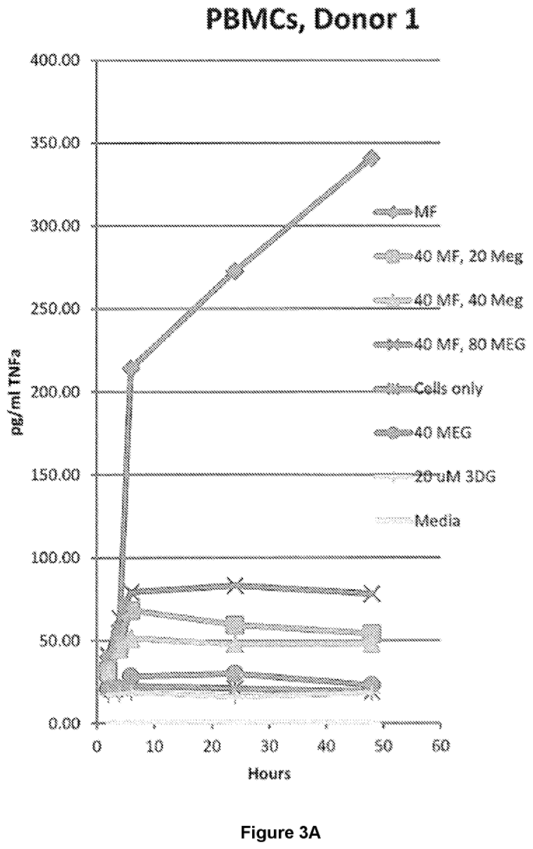

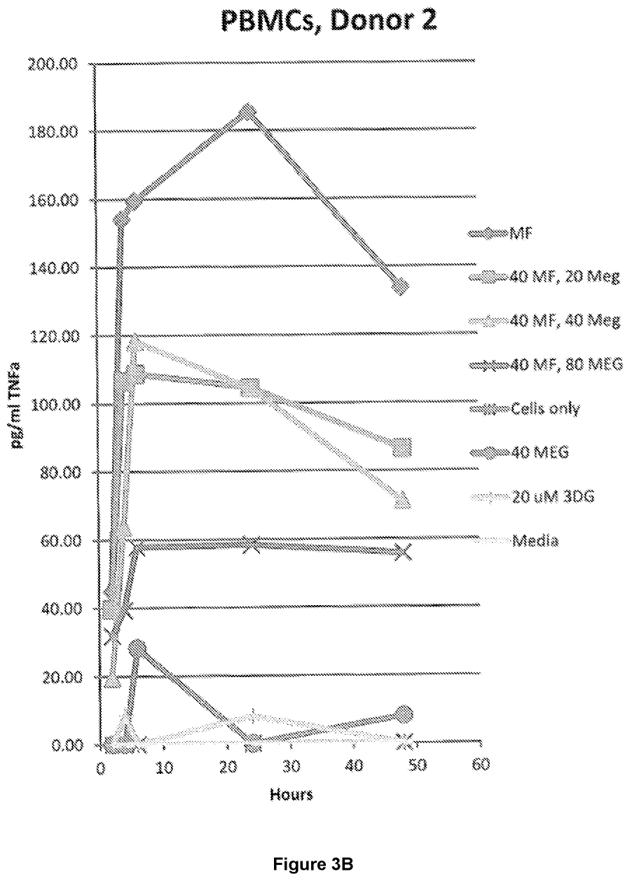

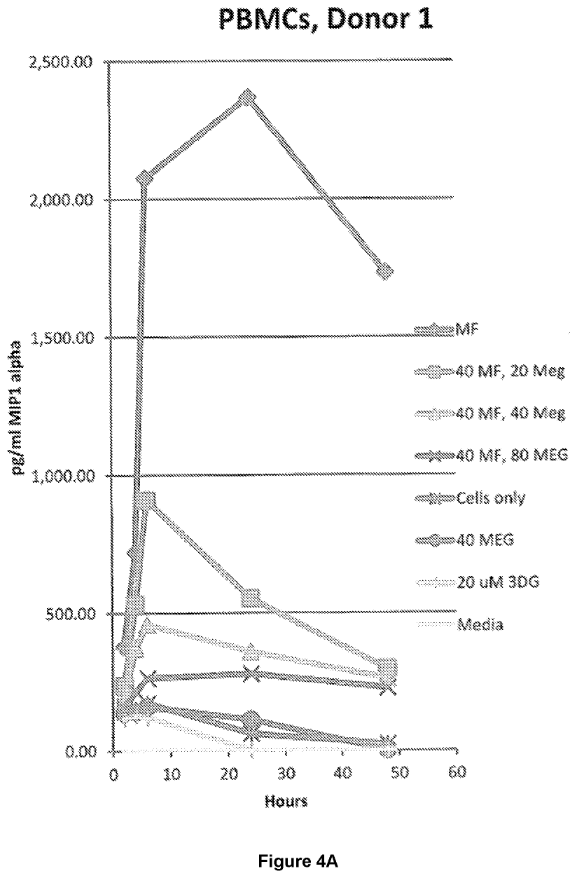

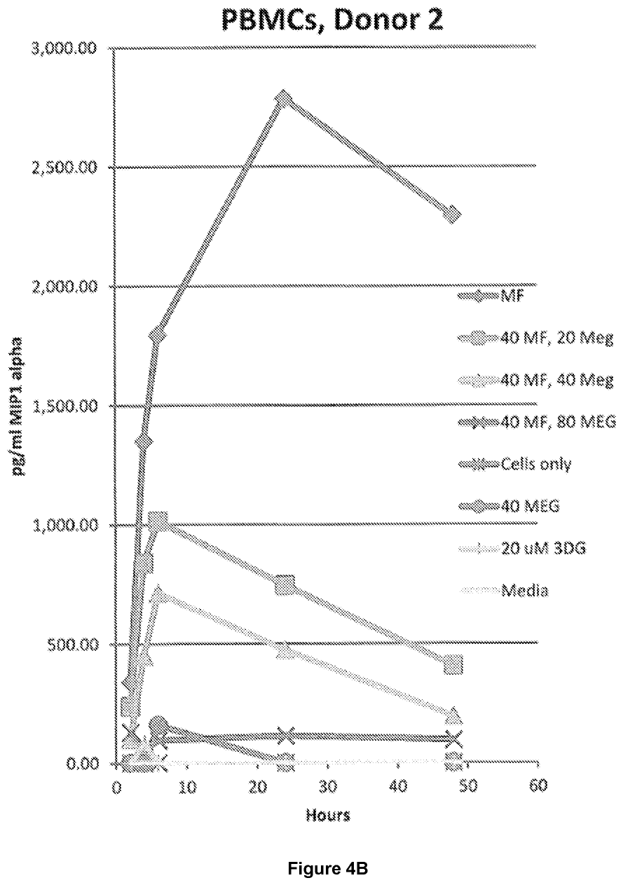

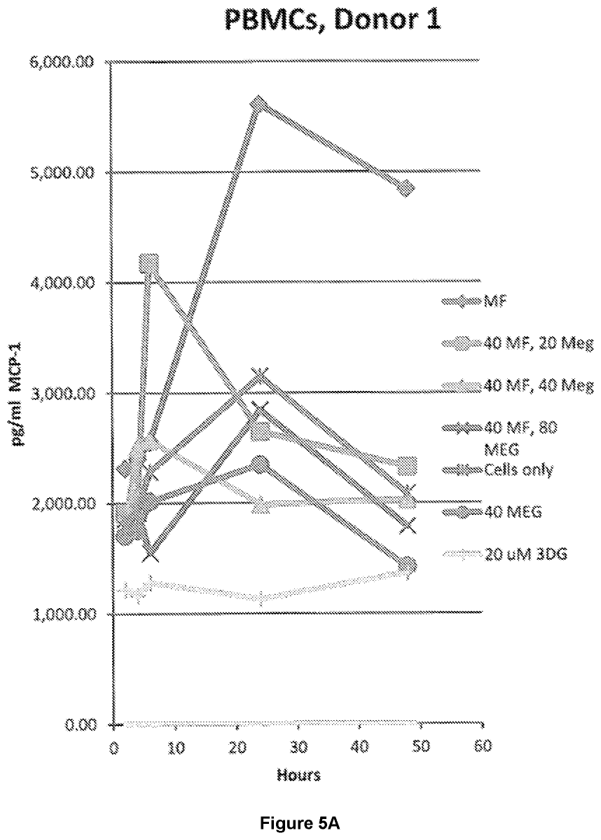

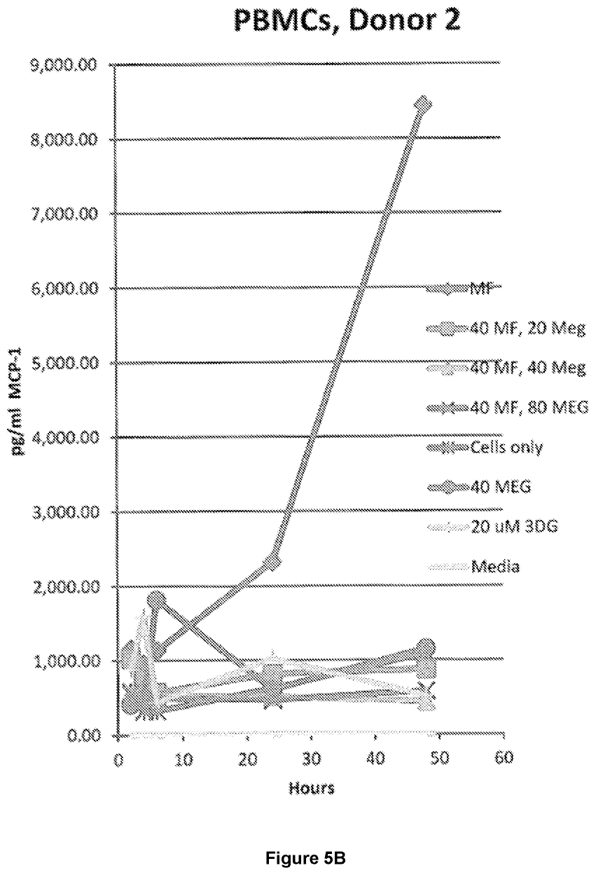

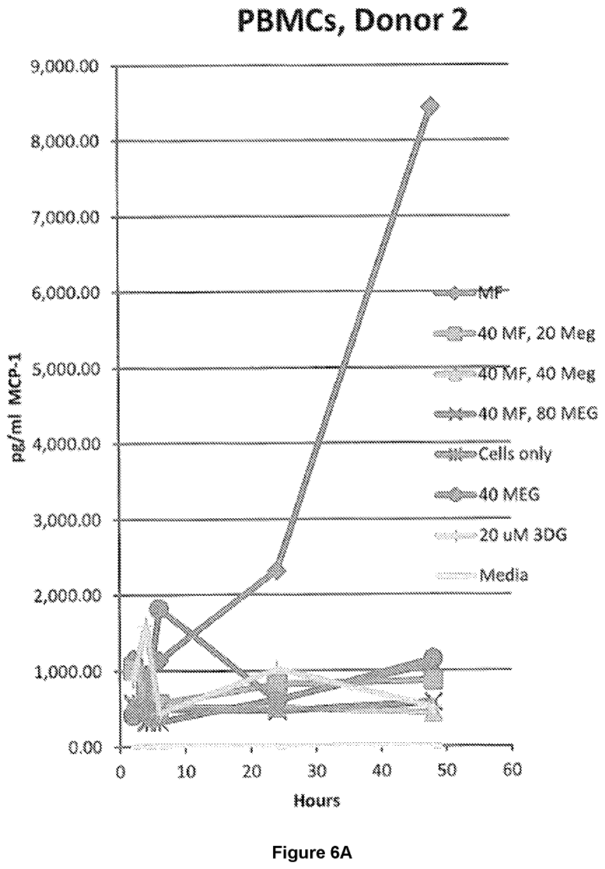

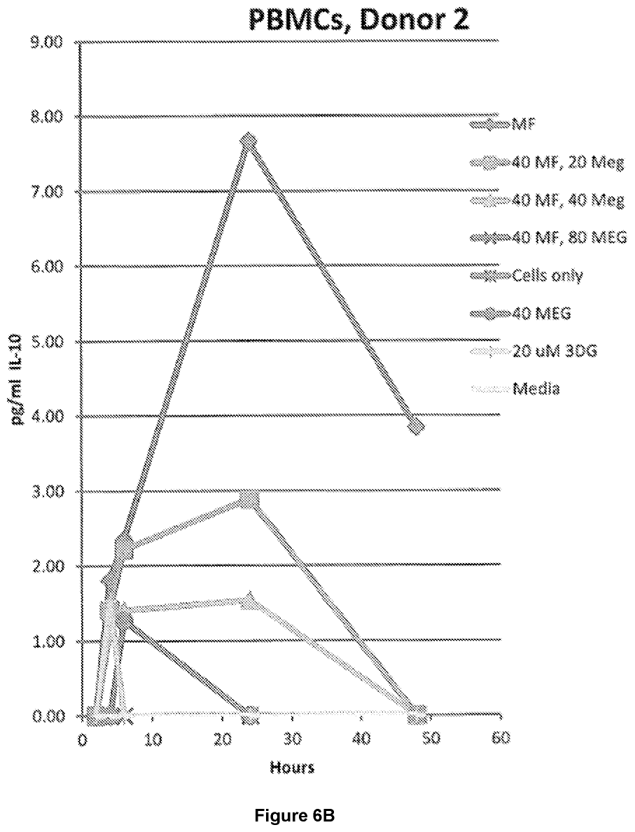

The same protocol was used to measure the addition of meglumine to reduce the levels of TNF-α (FIGS. 3A and 3B), MIP-1α (FIGS. 4A and 4B), MCP-1(FIGS. 5A and 5B), and MCP-1 ((FIGS. 6A and 6B).

### Example 2

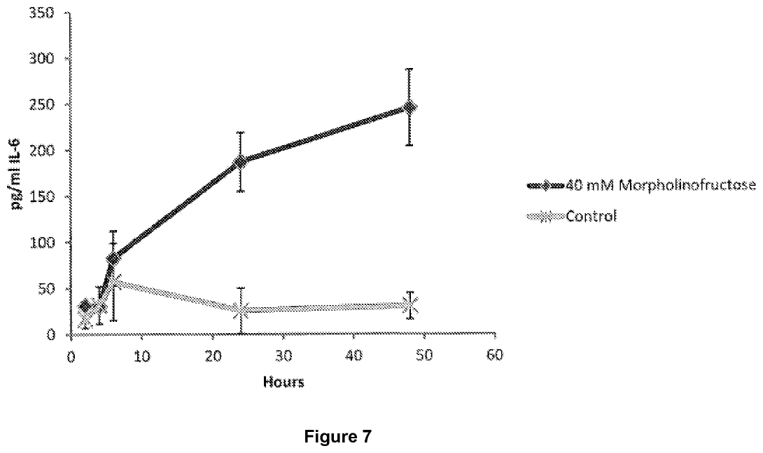

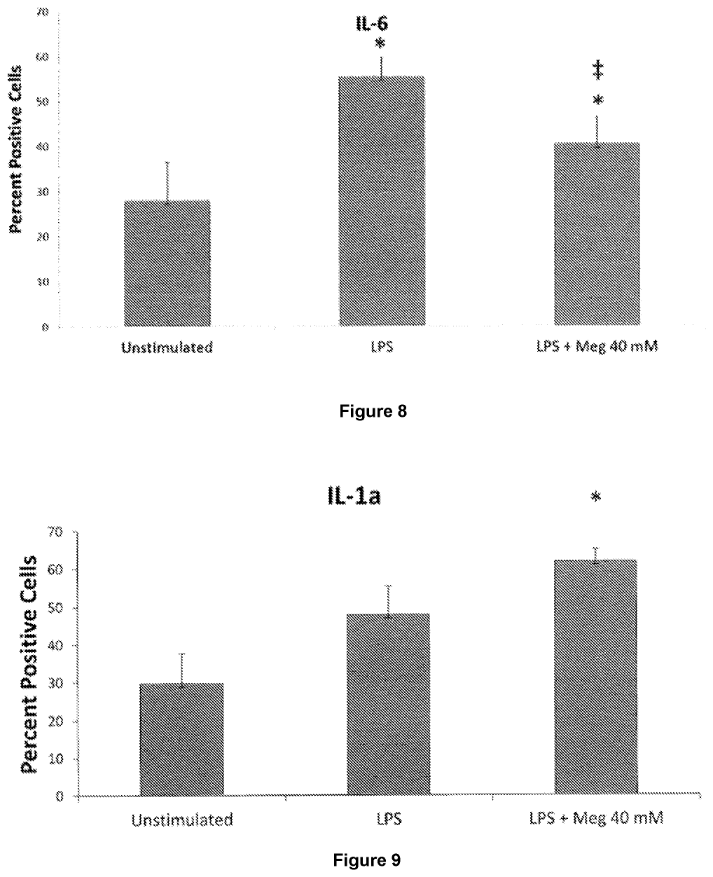

Lipopolysaccharide (LPS) at 40 mM concentration was used as a positive control for cytokine production in PBMCs. Meglumine was added at 40 mM and the cells allowed to incubate for 4 hours before sorting. Meglumine significantly reduced the LPS-stimulated increase in the percentage of IL-6 positive PBMCs, but did not return the cells to normal range. (p<0.05 vs unstimulated and p<0.05 vs LPS). FIG. 8. The same protocol was sued to measure IL-1α production. LPS and the combination of LPS and meglumine both significantly increased the percentage of PMBCs expressing IL-1α. FIG. 9.

### Example 3

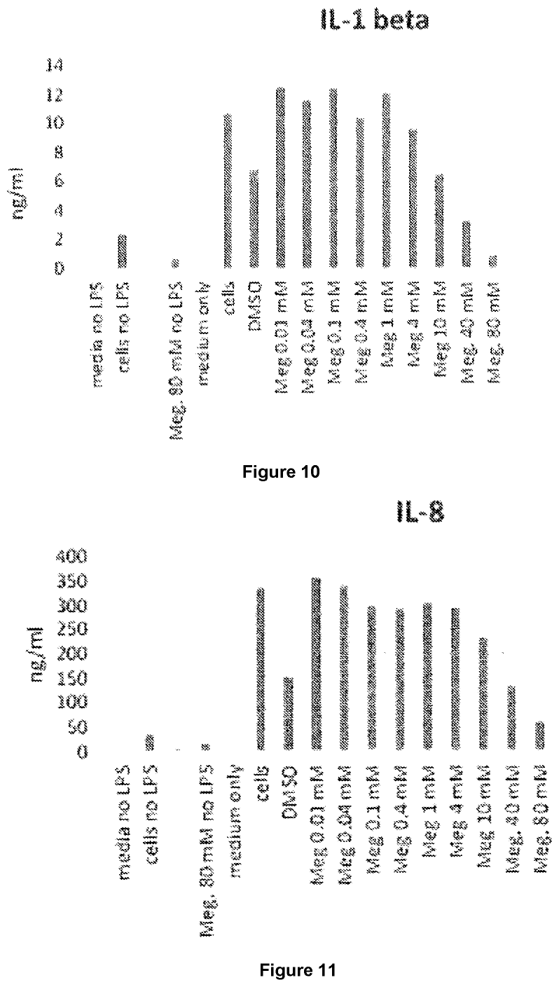

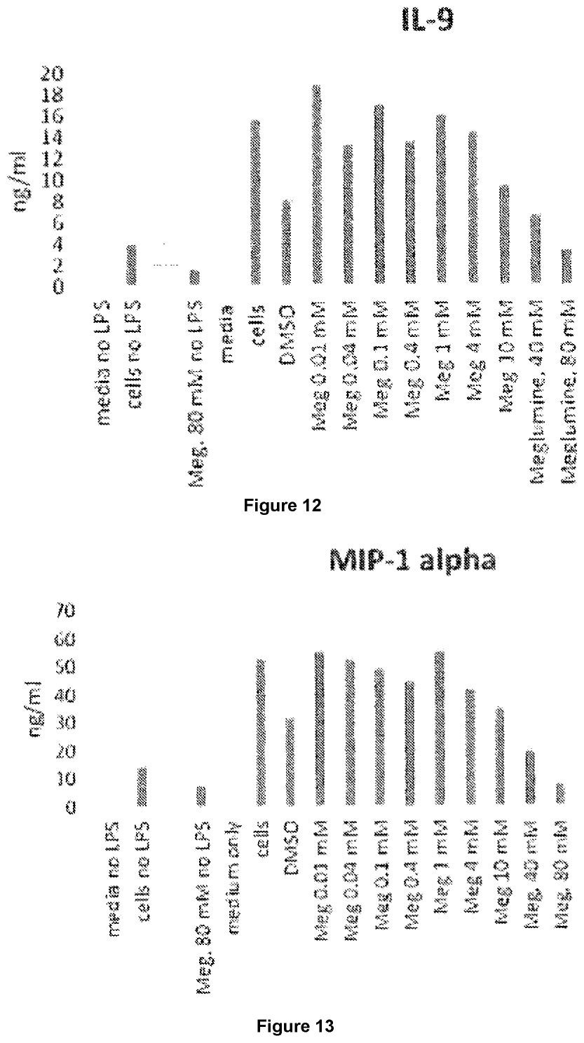

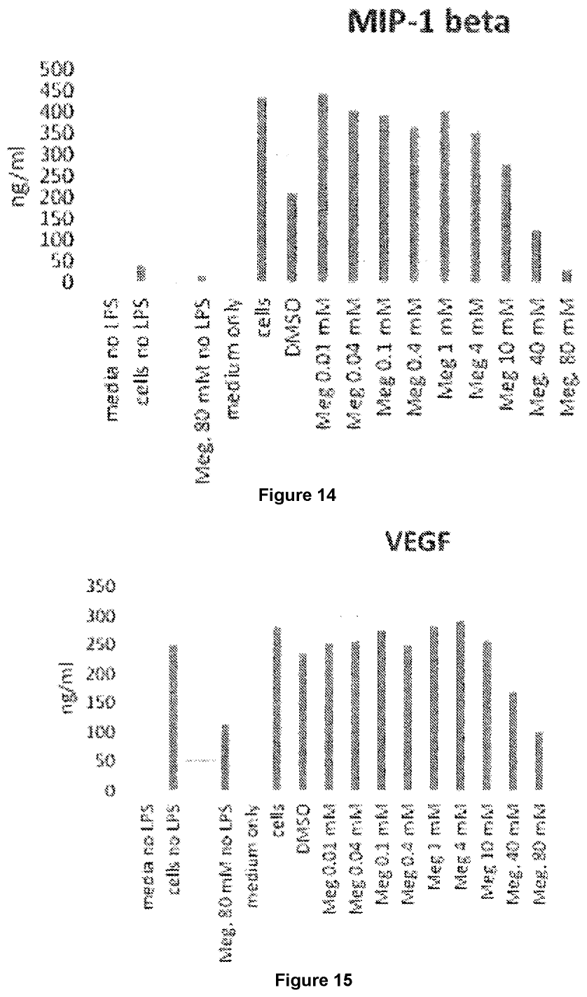

THP-1 cells (a human monocytic cell line derived from an acute monocytic leukemia patient) were cultured to 80% confluence. The THP-1 cells were then treated with LPS at 25 ng/ml to induce cytokine secretion. Cells were treated with either various concentration of meglumine. Tissue culture media was harvested at 24 hours and the levels of cytokines were determined. Meglumine, at high concentrations, displayed the ability to interfere with the secretion of the following cytokines: IL-1β (FIG. 10), IL-8 ((FIG. 11), IL-9 (FIG. 12), MIP-1α (FIG. 13), MIP-1β (FIG. 14), and VEGF (FIG. 15). Cytokine reduction was exhibited at concentrations as low as 10 mm, but reductions at higher concentrations were more pronounced.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating a disorder in a subject wherein said disorder is brought about, at least in part, by a cytokine storm in the subject, wherein the method comprises administering to the subject a composition which comprises a therapeutically effective amount of meglumine or a salt thereof.

2. The method of claim 1, wherein the composition is administered to the subject by a route selected from the group consisting of inhalational, oral, rectal, vaginal, parenteral, topical, transdermal, pulmonary, subcutaneous, intranasal, buccal, ophthalmic, intrathecal, parenteral, intravenous, and any combinations thereof.

3. The method of claim 1, wherein the composition is administered to the subject at a frequency selected from the group consisting of once a day, twice a day, three times a day, four times a day, once a week, twice a week, three times a week, four times a week, once a month, twice a month, and any combinations thereof.

4. The method of claim 1, wherein the composition is administered to the subject as a controlled-release formulation.

5. The method of claim 1, wherein said cytokine storm was initiated by a disorder chosen from the group consisting of bacterial infections, viral infections, non-infectious conditions, genetic mutations, and therapeutic activity.

6. The method of claim 5, wherein:
the bacterial infection is selected from the group consisting of streptococcus, staphylococcus, and pancreatitis; or
the viral infection is selected from the group consisting of influenza viruses, human corona viruses, other non-human corona viruses, Epstein Barr virus, virus variola, dengue virus, Ebola virus, West Nile virus, hepatitis A virus, hepatitis B virus, hepatitis C virus, respiratory viruses, herpes cytomegalovirus and any mutation of any of the foregoing; or
the non-infectious condition is selected from the group consisting of graft-versus-host disease, (“GVHD”), multiple sclerosis, multiple organ dysfunction syndrome, fibrosis, lung injuries, rheumatic diseases, and blood cancers; or
the therapeutic activity is selected from the group consisting of mAb therapy and CAR-T.

7. (canceled)

8. The method of claim 6, wherein the influenza virus is selected from the group consisting of H1N1, H5N1, wine influenza, Spanish influenza, and avian (bird) influenzas, and any mutation of any of the foregoing; or the corona virus is selected from the group consisting of 229E, NL63, OC43, HKU1, MERS-CoV, SARS-CoV, and SARS-CoV2, and any mutation of any of the foregoing.

9-11. (canceled)

12. The method of claim 1, further comprising administering to the subject the composition which comprises meglumine or a salt thereof by a route selected from the group consisting of inhalational, oral, rectal, vaginal, parenteral, topical, transdermal, pulmonary, intranasal, buccal, ophthalmic, intrathecal, parenteral, intravenous, subcutaneous, intramuscular, and any combinations thereof.

13. The method of claim 1, wherein the invention comprises administering meglumine, or a salt thereof, is administered in an amount equal to the amount of meglumine contained in an accepted dosage of DOTAREM® (gadoterate-meglumine).

14. The method of claim 13, wherein the accepted FDA approved dose for intravenous for intravenous administration is a dose that contains 0.9 grams of meglumine (ionically bound to the gadoterate) in a 10 ml solution.

15. The method of claim 14, wherein the method does not include the administration of the gadoterate portion of DOTAREM.

16. The method of claim 1, wherein the invention comprises intravenously administering meglumine, or a salt thereof, at between an amount between about 0.2 grams to about 10 grams per dose.

17. The method of claim 16, wherein such dose is administered during a period of once per hour to about once per six-hour period.

18. The method of claim 1, wherein the invention comprises orally administering meglumine, or a salt thereof at between an amount between about 0.2 grams to about 4 grams per dose.

19. The method of claim 1, wherein the invention comprises transdermally administering meglumine, or a salt thereof at a release level of between an amount between about 0.5 grams to about 3 grams per patch.

20. A pharmaceutical composition for treating a condition in a subject wherein said condition is brought about, at least in part, by a cytokine storm in the subject, wherein said composition comprises a pharmaceutically acceptable excipient and a therapeutically effective amount of meglumine or a salt thereof, in a dosage range of between about 10 mg per kg body weight and about 200 mg per kg body weight.

21. The pharmaceutical composition of claim 20, formulated for intravenous injection or infusion.

22. The pharmaceutical composition of claim 21, comprising between about 80 mg/ml and about 120 mg/ml of meglumine in a buffered saline solution.

23. (canceled)

24. The pharmaceutical composition of claim 20, consisting essentially of meglumine as an active ingredient.

25. The pharmaceutical composition of claim 20, consisting of meglumine as an active ingredient.
